(19) United States
(12) Patent Application Publication
(10) Pub. No.: US 2025/0268781 A1
(43) Pub. Date: Aug. 28, 2025

(54) CONTROL METHOD, DEVICE, TERMINAL, AND STORAGE MEDIUM OF EXTERNAL EAR NEGATIVE PRESSURE GENERATOR

(71) Applicant: Ningbo Albert Novosino Co., Ltd., Ningbo (CN)

(72) Inventors: Yonggui ZHANG, Ningbo (CN); Haibo HU, Ningbo (CN)

(21) Appl. No.: 18/584,013

(22) Filed: Feb. 22, 2024

(51) Int. Cl. A61H 21/00; A61F 11/08; A61H 9/00

(52) U.S. Cl. CPC A61H 21/00; A61F 11/08; A61H 9/0007; A61H 9/0057; A61H 9/0071

(57) ABSTRACT

A control method includes: obtaining a start information of the external ear negative pressure generator; obtaining an triggering information based on the start information; generating a negative pressure information based on the triggering information; obtaining a pressure detection information; judging whether the pressure detection information is smaller than or equal to a pressure value corresponding to a preset safety pressure information; continuing to obtain the pressure detection information, if the above judgment result is small; obtaining a pressurization triggering information, if the above judgment result is equal; judging whether the pressurization triggering information meets a requirement of a preset baseline pressurization triggering information; controlling the negative pressure device to stop generating the negative pressure information, if the judgment result is not desired; and controlling the negative pressure device to perform pressurization by a preset pressurization data information according to the pressurization triggering information, if the above judging result is desired.

### TECHNICAL FIELD

The present application relates to a field of medical equipment technology, and in particular, to a control method, a device, a terminal and a storage medium of an external ear negative pressure generator.

### BACKGROUND ART

An external ear negative pressure generator is a medical device configured to relieve migraine caused by the trigeminal electrical storm in such a way that the external auditory meatus negative pressure stimulates the ear drum and the external auditory meatus wall (trigeminal nerve and the ARNOLD nerve of the auriculotemporal branch) to balance the bioelectricity energy.

In an existing technology, the external ear negative pressure generator generally includes an ear plug, an gas tube and a gasbag, wherein the gasbag is fixed on an end of the gas tube, the gas tube is in communication with the gasbag, the ear plug is fixed on an end of the gas tube departing from the gasbag, the ear plug is inserted in the external auditory meatus to form a confined space, thereby compressing the gasbag to generate a gas. The air enters the confined space of the external auditory meatus through the gas tube to generate a negative pressure, thereby pressing the ear drum and simulating a branch nerve of the trigeminal nerve in the ear.

Regarding the above existing technology, the gas amount generated by pressing the gasbag is not stable, and the gas enters the confined space of the external auditory meatus to form the negative pressure by manually pressing the gasbag. This easily leads to excessive gas entering the confined space of the external auditory meatus, and generating excessive negative pressure to puncture the ear drum. Therefore, the safety during the use of the negative pressure generator on the external auditory meatus is low, and there is some spaces for improvement.

### SUMMARY

In order to improve safety during use of an external ear negative pressure generator, a control method, a device, a terminal and a storage medium of the external ear negative pressure generator in the present application is disclosed.

In the first aspect, a method for controlling the external ear negative pressure generator according to the present application adopts following solution:
a method for controlling an external ear negative pressure generator, comprising:
obtaining a start information of the external ear negative pressure generator;
obtaining an external auditory meatus triggering information based on the start information;
controlling a preset negative pressure device to generate a negative pressure information based on the external auditory meatus triggering information, and obtaining a pressure detection information of an external auditory meatus;
judging whether a value of the pressure detection information is smaller than or equal to a pressure value corresponding to a preset safety pressure information;
continuing to obtain the pressure detection information, if the value of the pressure detection information is smaller than the pressure value corresponding to the preset safety pressure information; or
obtaining a pressurization triggering information if the value of the pressure detection information is equal to the pressure value corresponding to the preset safety pressure information;
judging whether the pressurization triggering information meets a requirement of a preset baseline pressurization triggering information; and
controlling the negative pressure device to stop generating the negative pressure information, if the pressurization triggering information does not meet the requirement of the preset baseline pressurization triggering information; or
controlling the negative pressure device to perform pressurization by a preset pressurization data information according to the pressurization triggering information, if the pressurization triggering information meets the requirement of the preset baseline pressurization triggering information.

By adopting above solution, when the external ear negative pressure generator is using, the pressure detection information of the external auditory meatus is detected, so as to compare the pressure detection information with the safety pressure information. If the pressure detection information is smaller than the safety pressure information, it means that the pressure of the external auditory meatus is in a safety range, so as to continuously detect the pressure detection information. If the pressure detection information is equal to the safety pressure information, it means that the pressure of the external auditory meatus reaches the safety threshold, so as to detect the pressurization triggering information, and judge whether to perform pressurization by the pressurization triggering information, which may avoid an injury for the external auditory meatus of the operator by an excessive pressure of a pressurization for one time, thereby improving the safety when the external ear negative pressure generator is using.

In the second aspect, a device according to the present application adopts following solution:
the device used with any one of above methods for controlling an external ear negative pressure generator, comprising:
an ear plug configured to insert into the external auditory meatus to form a confined space where the negative pressure device generates the negative pressure information;
a negative pressure device connected to the ear plug, and configured to generate the negative pressure information;
a detecting device provided on the ear plug, and configured to detect the detected negative pressure information; and
a processing device electrically connected to the negative pressure device and the detecting device, respectively, and configured to compare the negative pressure information with the safety negative pressure information, and control the negative pressure device to generate the negative pressure information; and control the detecting device to detect the detected negative pressure information according to a comparison result.

By adopting above solution, the ear plug is inserted into the external auditory meatus to form a confined space, such that the processing device controls the negative pressure device to generate the negative pressure information in the confined space, the detecting device detects the negative pressure information, and, after the processing device compares the negative pressure information with the safety negative pressure information, the negative pressure device is controlled to generate or stop generating the negative pressure information, thereby improving the safety when the external ear negative pressure generator is using.

In the third aspect, an intelligent terminal according to the present application adopts following solution:
the intelligent terminal, comprising a memory and a processor, wherein the memory stores a computer program capable of being loaded with and executing any one of above methods for controlling the external ear negative pressure generator by the processor.

By adopting above solution, the processor loads with and executes the computer program of any one of above methods for controlling the external ear negative pressure generator stored in the memory by operating the intelligent terminal, thereby controlling the external ear negative pressure generator, and improving the safety when the external ear negative pressure generator is using.

In the fourth aspect, a computer-readable storage medium according to the present application is disclosed, which can store the related program, and is conducive to realizing improving the safety when the external ear negative pressure generator is using. The computer-readable storage medium adopts following solution:

a computer-readable storage medium is stored with a computer program being loaded and being implemented any one of above methods for controlling the external ear negative pressure generator by the processor.

By adopting above solution, the computer program of the methods for controlling the external ear generator is stored in the computer-readable storage medium, and the computer program in the storage medium is loaded with and executed by the process, thereby completing the control for the external ear negative pressure generator, and improving the safety when the external ear negative pressure generator is using.

In summary, the present application has at least one of the following beneficial technical effects:
1. the present application can avoid an injury to an external auditory meatus of the operator due to an excessive pressure of a pressurization for one time, thereby improving a safety when the external ear negative pressure generator is using;
2. the present application can control a negative pressure device to generate a negative pressure information according to a pressurization data information, which is based on a base negative pressure information, thereby improving an efficiency of a negative pressure device for generating the negative pressure information; and
3. the present application can obtain a deformation length information by calculating a changed capacity information and an external auditory meatus diameter information, such that the negative pressure device drives an ear plug to deform by the deformation length information, thereby changing a capacity of an external auditory meatus, compressing a gas in the external auditory meatus to generate a base negative pressure information, and improving a convenience for generating the base negative pressure information.

### BRIEF DESCRIPTION OF THE DRAWINGS

FIG. 1 is a flow chat of a control method of an external ear negative pressure generator according to an embodiment of the present application.

FIG. 2 is a flow chat of a method for controlling a preset negative pressure device to generate a negative pressure information based on the external auditory meatus triggering information according to an embodiment of the present application.

FIG. 3 is a flow chat of a method for controlling the negative pressure device to generate a base negative pressure information based on a safety pressure information according to an embodiment of the present application.

FIG. 4 is a flow chat of a method for controlling the negative pressure device to drive an ear plug to deform by a deformation length corresponding to a deformation length information and compress a gas to generate the base negative pressure information according to an embodiment of the present application.

FIG. 5 is a flow chat of a method for controlling the negative pressure device to deform the ear plug and to generate the base negative pressure information based on a safety deformation length information according to an embodiment of the present application.

FIG. 6 is a flow chat of a depressurizing method of the negative pressure information according to an embodiment of the present application.

FIG. 7 is a flow chat of a control method of the negative pressure information according to an embodiment of the present application.

FIG. 8 is a sectional schematic view of the external ear negative pressure generator according to an embodiment of the present application;

### DETAILED DESCRIPTION

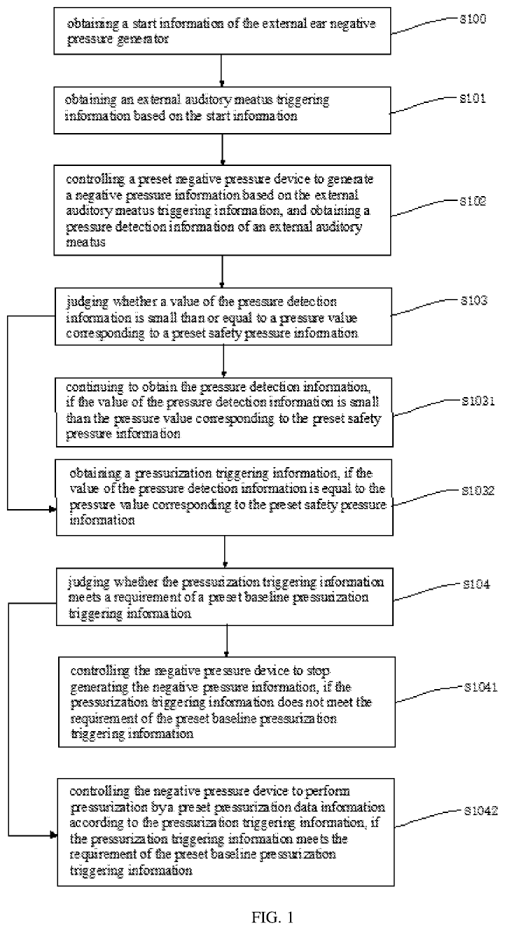

In order to illuminate the purpose and the technical solution of present application more clearly, the present application is further described in detail below in combination with FIGS. 1-8. It should be understood that, the detail embodiment herein is only used for explaining the present application, but not used for limiting the present application.

Referring to FIG. 1, a control method of an external ear negative pressure generator in the present application includes following steps:

Step 100: obtaining a start information of the external ear negative pressure generator.

In particular, the start information is a voltage in an electric loop of the external ear negative pressure generator when an operator presses a power switch of the external ear negative pressure generator. The operator presses the power switch to close the electric switch and start the external ear negative pressure generator.

Step 101: obtaining an external auditory meatus triggering information based on the start information.

In particular, the external auditory meatus triggering information is a triggering signal when the ear plug is inserted into the external auditory meatus of the operator after the external ear negative pressure generator is started, which is detected by a force sensitive sensor installed on the ear plug and sent to a processing device. A method for detecting is as follows: after the external ear negative pressure generator is started, the detecting result of the force sensitive sensor is retrieved, if the detecting result of the force sensitive sensor is larger than zero, it means that the ear plug is inserted into the external auditory meatus and compressed by the external auditory meatus, thereby outputting the external auditory meatus triggering information.

Step 102: controlling a preset negative pressure device to generate a negative pressure information based on the external auditory meatus triggering information, and obtaining a pressure detection information of the external auditory meatus.

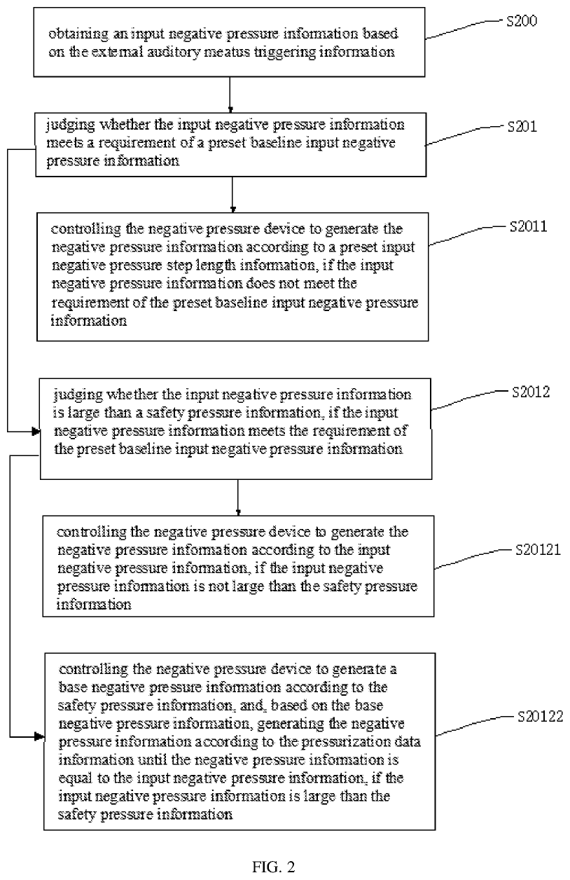

In particular, the negative pressure device is a device configured to generate the negative pressure in a confined space formed between the ear plug and the external auditory meatus, which can be composed of a piezoelectric ceramic pump and a gas tube, wherein the piezoelectric ceramic pump generates vibration to deliver the gas to the confined space through the gas tube or to deliver the gas to the ear plug for deforming the ear plug, thereby changing the negative pressure in the confined space. When the external auditory meatus triggering information is detected, the negative pressure device is controlled to generate the negative pressure information in the confined space. The detail generating method is referred to the steps in the FIG. 2.

The pressure detection information is a pressure value formed by the gas in the confined space of the external auditory meatus for the external auditory meatus and the ear drum, which is detected by a force sensitive sensor installed on the ear plug and sent to a processing device. The pressure of the gas in the external auditory meatus is monitored every time by detecting the pressure detection information, thereby avoiding an injury due to the excessive pressure.

Step 103: judging whether a value of the pressure detection information is smaller than or equal to a pressure value corresponding to a preset safety pressure information.

In particular, the safety pressure information is the largest pressure value that the external auditory meatuses from all age groups are not injured, which is specifically set by those skilled in the art according to actual situations.

It is determined whether the pressure value of the confined space of the external auditory meatus reached to a largest pressure value that the external auditory meatus is not injured by judging whether the pressure value corresponding to the pressure detection information is smaller than or equal to the pressure value corresponding to the safety pressure information.

Step 1031: continuing to obtain the pressure detection information, if the value of the pressure detection information is smaller than the pressure value corresponding to the preset safety pressure information.

In particular, if the pressure value corresponding to the pressure detection information is smaller than the pressure value corresponding to the safety pressure information, it means that the pressure value of the confined space of the external auditory meatus does not reach the largest pressure value that the external auditory meatus is not injured, and the negative pressure generated by the negative pressure device will not injure the external auditory meatus, thereby continuing to detect the pressure detection information and continuing to monitor the changing of the pressure detection information.

Step 1032: obtaining a pressurization triggering information if the value of the pressure detection information is equal to a pressure value corresponding to the preset safety pressure information.

In particular, the pressurization triggering information is a voice information of the operator, such as “continuing to pressurize” and other semantic features, recognized by the voice module of the external auditory meatus when the pressure value corresponding to the pressure detection information is equal to the pressure value corresponding to the safety pressure information.

Step 104: judging whether the pressurization triggering information meets a requirement of a preset baseline pressurization triggering information.

In particular, the baseline pressurization triggering information, such as “continuing to pressurize”, is a triggering information by controlling the negative pressure device to continue to generate the negative pressure information, which is set by those skilled in the art according to actual needs.

The requirement of the baseline pressurization triggering information is same as the semantic features corresponding to the baseline pressurization triggering information. It is determined whether the operator is necessary to continue to pressurize on the base of the pressure value corresponding to the safety negative pressure information by judging whether the semantic features corresponding to the pressurization triggering information is same as the semantic features corresponding to the baseline pressurization triggering information.

Step 1041: controlling the negative pressure device to stop generating the negative pressure information, if the pressurization triggering information does not meet the requirement of the preset baseline pressurization triggering information.

In particular, if the semantic features corresponding to the pressurization triggering information are different from the semantic features corresponding to the baseline pressurization triggering information, it means that the operator is not necessary to continue to pressurize on the base of the pressure value corresponding to the safety negative pressure information, whereby the negative pressure device is controlled to stop generating the negative pressure information.

Step 1042: controlling the negative pressure device to perform pressurization by a preset pressurization data information according to the pressurization triggering information, if the pressurization triggering information meets the requirement of the preset baseline pressurization triggering information.

In particular, the pressurization data information is a pressurization speed when the negative pressure device performs pressurization, which is set by those skilled in the art according to actual needs. If the semantic features corresponding to the pressurization triggering information are same as the semantic features corresponding to the baseline pressurization triggering information, it means that the operator has to continue to perform pressurization on the base of the pressure value corresponding to the safety negative pressure information, thereby controlling the negative pressure device to continue to perform pressurization by the pressurization speed corresponding to the pressurization data information.

Referring to FIG. 2, a method for controlling the preset negative device to generate the negative pressure information based on the external auditory meatus triggering information includes following steps:

Step 200: obtaining an input negative pressure information based on the external auditory meatus triggering information.

In particular, the input negative pressure information is a detection data of the input negative pressure value of the processing device for the operator when the ear plug is inserted into the external auditory meatus, which includes two situations, that is, the presence or absence of the input negative pressure value.

Step 201: judging whether the input negative pressure information meets a requirement of the preset baseline input negative pressure information.

In particular, the baseline input negative pressure information is a detection data of the operator's input negative pressure value detected by the processing device. The requirement of the baseline input negative pressure information is same as the detection data corresponding to the baseline input negative pressure information.

It determined that whether the operator inputs the negative pressure value as needed in the processing device by judging whether the detection data corresponding to the negative pressure information is same as the detection data corresponding to the negative pressure information.

Step 2011: controlling the negative pressure device to generate the negative pressure information according to a preset input negative pressure step length information, if the input negative pressure information does not meet the requirement of the preset baseline input negative pressure information,

In particular, the input negative pressure step length information is a negative pressure value generated by the negative pressure device in unit time when there is no input negative pressure value, which specific value is set by those skilled in the art according to actual needs. If the detection data corresponding to the input negative pressure information is different from the detection data corresponding to the baseline input negative pressure information, which means that the detection data corresponding to the input negative pressure information is a situation that there is no input negative pressure value, thereby controlling negative pressure device to input the negative pressure increment corresponding to the negative pressure increasing value in unit time.

Step 2012: judging whether a value of the input negative pressure information is larger than a value of the safety pressure information, if the input negative pressure information meets the requirement of the preset baseline input negative pressure information.

In particular, If the detection data corresponding to the input negative pressure information is same as the detection data corresponding to the baseline input negative pressure information, it means that the detection data corresponding to the input negative pressure information is a situation that there is the input negative pressure value, thereby judging whether the negative pressure value corresponding to the negative pressure information is larger than the pressure value corresponding to the safety pressure information, and determining whether to generating the negative pressure stepwise or for one time by the negative pressure.

Step 20121: controlling the negative pressure device to generate the negative pressure information according to the input negative pressure information, if the input negative pressure information is not larger than the safety pressure information.

In particular, if the negative pressure value corresponding to the negative pressure information is not larger than the pressure value corresponding to the safety pressure information, it means that the input negative pressure information does not injure the external auditory meatus of the operator, thereby controlling the negative pressure device to input the negative pressure value corresponding to the negative pressure information to generate the negative pressure information for one time.

Step 20122: controlling the negative pressure device to generate a base negative pressure information according to the safety pressure information, and, based on the base negative pressure information, generating the negative pressure information according to the pressurization data information until the negative pressure information is equal to the input negative pressure information, if the input negative pressure information is larger than the safety pressure information.

In particular, the base negative pressure information is a negative pressure value generated by the negative pressure device that is based on the safety pressure information. If the negative pressure value corresponding to the value pressure information is larger than the pressure value corresponding to the safety pressure information, it means that the input negative pressure information will injure the external auditory meatus of the operator, thereby controlling the negative pressure device to generate the base negative pressure information based on the pressure value corresponding to the safety pressure information, and based on the negative pressure value corresponding to the base negative pressure information, controlling the negative pressure device to perform pressurization by the pressurization spade which is corresponding to the pressurization data information until the negative pressure value corresponding to the negative pressure information is equal to the negative pressure value corresponding to the input negative pressure information.

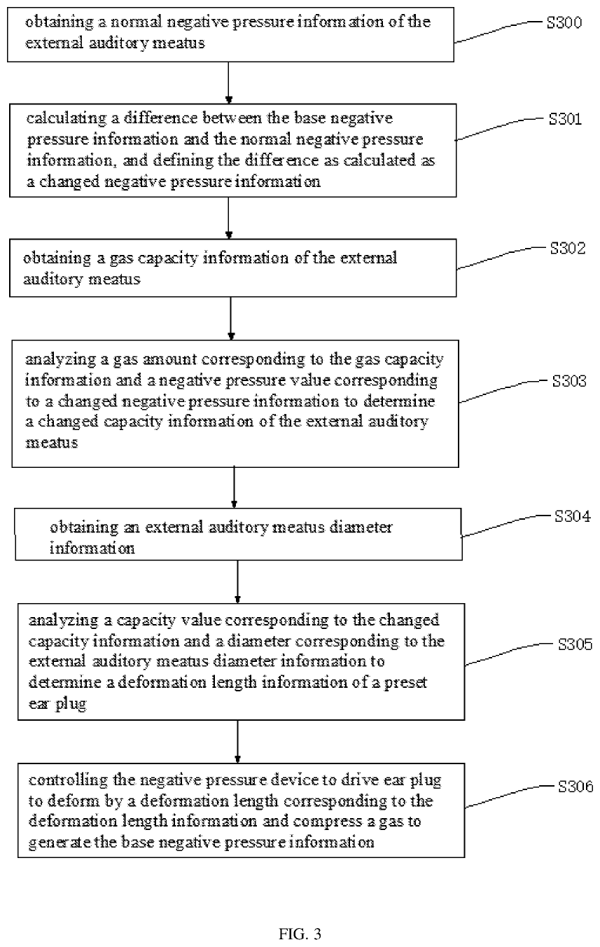

Referring to FIG. 3, a method for controlling the negative pressure device to generate the base negative pressure information according to the safety pressure information includes following steps:

Step 300: obtaining a normal negative pressure information of the external auditory meatus.

In particular, the normal negative pressure information is a pressure value of the gas in the external auditory meatus when the ear plug is inserted into the external auditory meatus, which is detected by the force sensitive sensor of the ear plug and sent to the processing device.

Step 301: calculating a difference between the base negative pressure information and the normal negative pressure information, and defining the difference as calculated as a changed negative pressure information.

In particular, the changed negative pressure information is a negative pressure value increased from the negative pressure value corresponding to the normal negative pressure information to the negative pressure value corresponding to the base negative pressure information, which is obtained by the processing device calling the base negative pressure information and the normal negative pressure information, and calculating the difference between the negative pressure value corresponding to the base negative pressure and the negative pressure value corresponding to the normal negative pressure value.

Step 302: obtaining a gas capacity information of the external auditory meatus.

In particular, the gas capacity information is a gas amount of the external auditory meatus, the capacity value of the external auditory meatus is same as the gas amount value. the operator inputs the capacity value of the external auditory meatus into the processing device, and the processing device determines the gas amount according to the capacity value of the external auditory, thereby obtaining the gas capacity information.

Step 303: analyzing the gas capacity corresponding to the gas capacity information and the negative pressure value corresponding to the changed negative pressure information to determine a changed capacity information of the external auditory meatus.

In particular, the changed capacity information is a capacity value which is necessary to be reduced that the gas capacity corresponding to the gas capacity information increases the negative pressure corresponding to the changed negative pressure information in the external auditory meatus, which is analysed by the processing device calling the gas capacity information and the changed negative pressure information. The method for analyzing is: when the gas amount value corresponding to the gas capacity information is constant, the negative pressure value corresponding to the changed negative pressure information is larger, the capacity value corresponding to the changed capacity information will be smaller, thus the changed negative pressure information is inversely proportional to the changed capacity information, and the inverse proportion coefficient is set by those skilled in the art according to actual needs.

Step 304: obtaining an external auditory meatus diameter information.

In particular, the external auditory meatus diameter information is a diameter value of the external auditory meatus, which is obtained by the operator inputting in the processing device.

Step 305: analyzing a capacity value corresponding to the changed capacity information and a diameter corresponding to the external auditory meatus diameter information to determine a deformation length information of the preset ear plug.

In particular, the ear plug is a device inserted into the external auditory meatus to form a confined space, which can be made of rubber, configured to be in communication with the negative pressure device. The deformation length information is a length value that the ear plug deforms in the external auditory meatus to increase the changed negative pressure information, which is obtained by the processing device calling the changed capacity information and the external auditory meatus diameter information. The specific analyzing method is: using a formula for calculating the volume of a column, inputting the capacity value corresponding to the changed capacity information and the diameter value corresponding to the external auditory meatus diameter information, thereby calculating the deformed length value, and obtaining the deformation length information.

Step 306: controlling the negative pressure device to drive ear plug to deform by a deformation length corresponding to the deformation length information and compress a gas to generate the base negative pressure information.

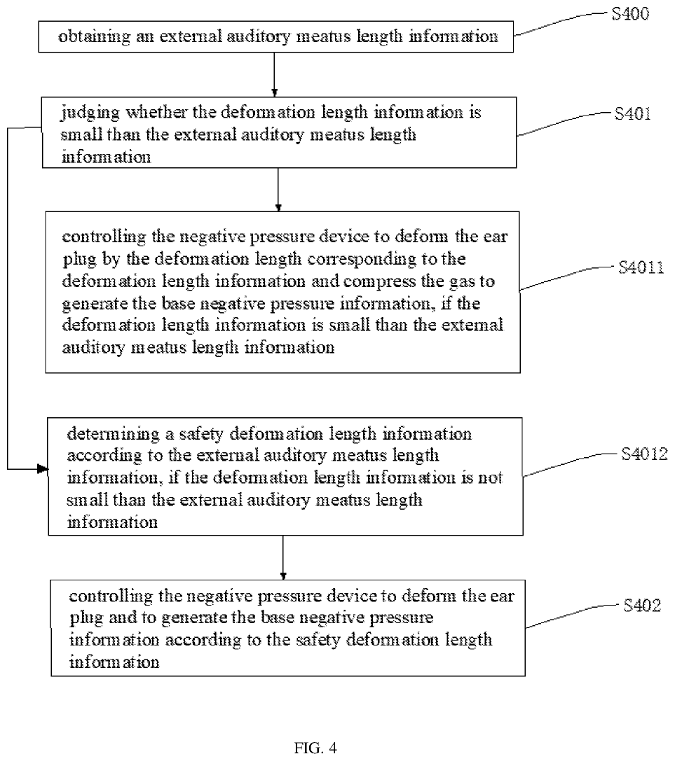

In particular, after determining the deformation length information, the negative pressure device is controlled to generate and deliver the gas to the ear plug, so that the ear plug is deformed by the length value corresponding to the deformation length information, thereby compressing the gas in the confined space, and forming the base negative pressure information. The specific method for driving the ear plug by the negative pressure device is referred to the steps in FIG. 4.

Referring to FIG. 4, a method for controlling the negative pressure device to drive ear plug to deform by a deformation length corresponding to the deformation length information and compress a gas to generate the base negative pressure information includes following steps:

step 400: obtaining an external auditory meatus length information.

In particular, the external auditory meatus length information is a length value of the external auditory meatus, which is obtained by the operator inputting into the processing device.

Step 401: judging whether a value of the deformation length information is smaller than a value of the external auditory meatus length information.

In particular, it is determined whether the length in the external auditory meatus reaches the lowest requirement of the deformation of the ear plug by judging whether the length value corresponding to the deformation length information is smaller than the length value corresponding to the external auditory meatus length information.

Step 4011: controlling the negative pressure device to deform the ear plug by the deformation length corresponding to the deformation length information and compress the gas to generate the base negative pressure information, if the value of the deformation length information is smaller than the value of the external auditory meatus length information.

In particular, if the deformation length information is smaller than the external auditory meatus length information, it means that the length in the external auditory meatus reaches the lowest deformation requirement of the ear plug, thereby controlling the negative pressure device to deform the ear plug by the deformation length corresponding to the deformation length information and compress the gas to generate the base negative pressure information. The specific deforming method is as follows: the negative pressure device generates the gas and delivers the gas to the ear plug, such that the gas deforms the ear plug, in which the different deformation length is corresponding to the different gas amount, the deformation length is directly proportional to the gas amount, and a direct proportion coefficient is set by those skilled in the art according to actual needs.

Step 4012: determining a safety deformation length information according to the external auditory meatus length information, if the deformation length information is not smaller than the external auditory meatus length information.

In particular, the safety deformation length information is a length value for deforming the ear plug based on the length value corresponding to the external auditory meatus length information when the deformation length information is not smaller than the external auditory meatus length information, a length value corresponding to the safety deformation length information is smaller than the length value corresponding to the external auditory meatus length information, and the proportion between the safety deformation length information and the external auditory meatus length information is set by the skill in the art according to actual needs.

Step 402: controlling the negative pressure device to deform the ear plug so as to generate the base negative pressure information according to the safety deformation length information.

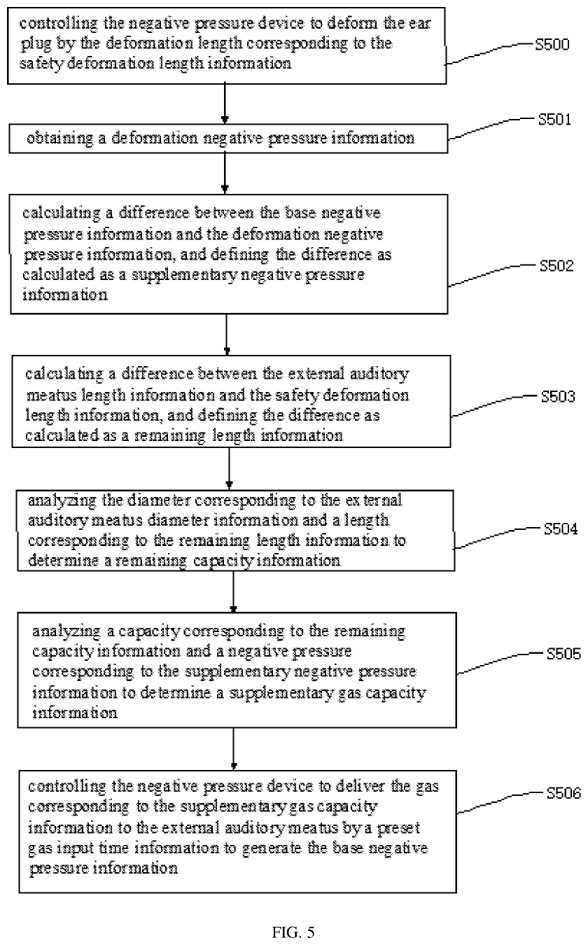

In particular, after determining the safety deformation length information, the negative pressure device is controlled to deform the ear plug by the length value corresponding to the safety deformation length information and compress the air to generate the base negative pressure information. The specific method for driving the negative pressure device is referred to the steps in FIG. 5.

Referring to FIG. 5, a method for controlling the negative pressure device to deform the ear plug so as to generate the base negative pressure information according to the safety deformation length information includes following steps:

Step 500: controlling the negative pressure device to deform the ear plug by the deformation length corresponding to the safety deformation length information.

In particular, the negative pressure device is controlled to deform the ear plug by the deformation length corresponding to the safety deformation length information, thereby compressing the gas in the confined space. The specific driving method is as follows: the negative pressure device generates the gas and delivers the gas to the ear plug, such that the gas deforms the ear plug, the different deformation length is corresponding to the different gas amount, the deformation length is directly proportion to the gas amount, and the direct proportion coefficient is set by the skill in the art according to actual needs. When the direct proportion and the deformation length are determined, the gas amount is calculated, such that the negative pressure device delivers the gas to the ear plug by the gas amount and compress the ear plug.

Step 501: obtaining a deformation negative pressure information.

In particular, the deformation negative pressure information is a pressure value of the gas in the confined space for the external auditory meatus and the ear drum after the ear plug is deformed by the length value corresponding to the safety deformation length information, which is detected by the force sensitive sensor and sent to the processing device.

Step 502: calculating a difference between the base negative pressure information and the deformation negative pressure information, and defining the difference as calculated as a supplementary negative pressure information.

In particular, the supplementary negative pressure information is a difference between the pressure of the gas in the confined space and the necessary pressure value when the length of the external auditory meatus does not reach the lowest requirement of the deformation length of the ear plug and after a part of the ear plug deforms, which is obtained by the processing calling the base negative pressure information and the deformation negative pressure information, and calculating the difference between the negative pressure value corresponding to the base negative pressure information and the negative pressure value corresponding to the deformation negative pressure information.

Step 503: calculating a difference between a value of the external auditory meatus length information and a value of the safety deformation length information, and defining the difference as calculated as a remaining length information.

In particular, the remaining length information is a remaining length value of the confined space in the external auditory meatus when the ear plug deforms in the external auditory meatus, which is obtained by the processing device calling the external auditory meatus information and the safety deformation length information, and calculating the difference between the length value corresponding to the external auditory meatus length information and the length value corresponding to the safety deformation length information.

Step 504: analyzing the diameter corresponding to the external auditory meatus diameter information and a length corresponding to the remaining length information, so as to determine a remaining capacity information.

In particular, the remaining capacity information is a remaining capacity value of the confined space of the external auditory meatus after the ear plug deforms, which is obtained by the processing device calling the external auditory information and the remaining length information. The calculating method includes calculating the capacity from input length and diameter by using a volume calculating formula.

Step 505: analyzing a volume corresponding to the remaining capacity information and a negative pressure corresponding to the supplementary negative pressure information to determine a supplementary gas capacity information.

In particular, the supplementary gas capacity information is a gas amount required to be added for increasing the negative pressure corresponding to the supplementary negative pressure information in the remaining confined space, which is analyzed by the processing device calling the remaining capacity information and the supplementary negative pressure information. The analyzing method is as follows: when the capacity value corresponding to the remaining capacity information is constant, the negative pressure value corresponding to the supplementary negative pressure information is directly proportional to the gas amount corresponding to the supplementary gas capacity information, in which the direct proportion coefficient is set by those skilled in the art according to actual needs.

Step 506: controlling the negative pressure device to deliver the gas corresponding to the supplementary gas capacity information to the external auditory meatus by a preset gas input time information to generate the base negative pressure information.

In particular, the gas input time information is a time value delivering the gas to the confined space, and the time is obtained by the operator inputting to the processing device. After determining the supplementary gas amount, the negative pressure device is controlled to deliver the gas of the gas amount corresponding to the supplementary gas capacity information by the gas input time information to the confined space of the external auditory meatus, such that the negative pressure value corresponding to the deformation negative pressure information is supplied after the ear plug deforms by the length corresponding to the safety deformation length information, thereby obtain the base negative pressure information.

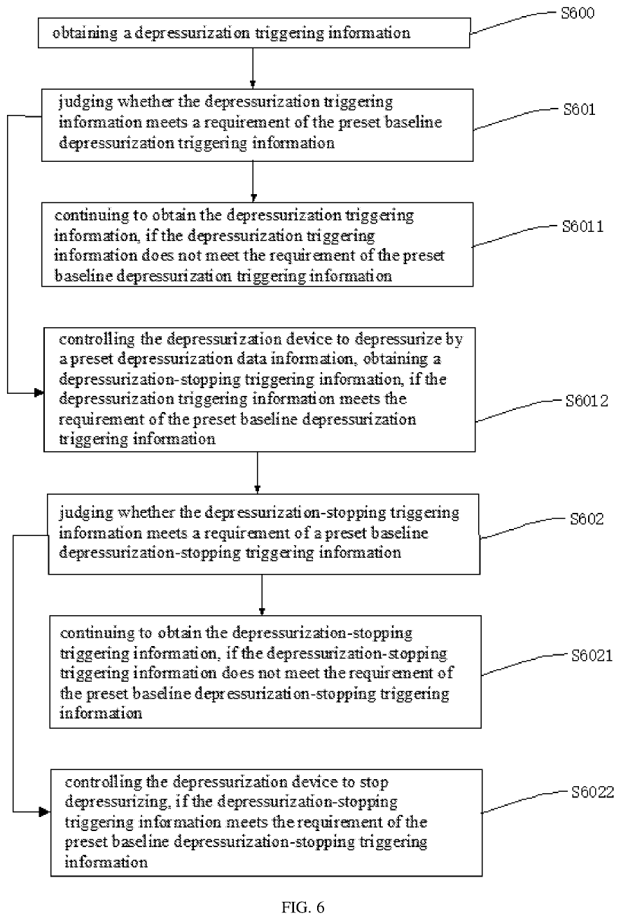

Referring to FIG. 6, when the negative pressure information is equal to the input negative pressure information, a depressurizing method of the negative pressure information includes following steps:

step 600: obtaining a depressurization triggering information.

In particular, the depressurization triggering information is a detection data of the operator's voice after the negative pressure device generates the negative pressure information, which is identified by a voice module of the external ear negative pressure generator.

Step 601: judging whether the depressurization triggering information meets a requirement of a preset baseline depressurization triggering information.

In particular, the base depressurization triggering information is a voice feature, such as a painful voice from the operator or a voice containing a semantic feature of depressurization from the operator, by which the depressurization device is controlled to depressurize the gas in the confined space. The requirement of the baseline depressurization triggering information is same as the voice feature corresponding to the basic depressurization triggering information.

It is determined whether the operator is necessary to reduce the negative pressure value of the confined space of the external auditory meatus by judging whether the voice detection data corresponding to the depressurization triggering information is same as the voice feature corresponding to the baseline triggering information.

Step 6011: continuing to obtain the depressurization triggering information, if the depressurization triggering information does not meet the requirement of the preset baseline depressurization triggering information.

In particular, if the voice detection data corresponding to the depressurization triggering information is different from the voice feature corresponding to the baseline triggering information, it means that the operator does not needs to reduce the negative pressure value of the confined space of the external auditory meatus. Therefore, the depressurization triggering information is continuously detected, thereby continuing to monitor the changes in the depressurization triggering information.

Step 6012: controlling the depressurization device to perform depressurization by a preset depressurization data information, and obtaining a depressurization-stopping triggering information, if the depressurization triggering information meets the requirement of the preset baseline depressurization triggering information.

In particular, the depressurization data information is a speed value of the gas discharged from the confined space of the external auditory meatus, which is set by those skilled in the art according to actual needs. The depressurization device is a device configured to discharge the gas in the confined space of the external auditory meatus, wherein the depressurization device is in communication with the gas tube of the negative pressure device, the depressurization device can be an electrical valve, and the depressurization device is controlled by the processing device. The depressurization-stopping triggering information is a voice detection data of the operator identified by the voice recognition module of the external ear negative pressure generator when the depressurization device discharges the gas in the confined space of the external auditory meatus.

If the voice detection data corresponding to the depressurization triggering information is same as the voice feature corresponding to the baseline triggering information, it means that the operator is necessary to reduce the negative pressure value of the confined space of the external auditory meatus, thereby controlling the depressurization device to discharge the gas in the confined space of the external auditory meatus by the depressurization speed value corresponding to the depressurization data information.

Step 602: judging whether the depressurization-stopping triggering information meets a requirement of a preset baseline depressurization-stopping triggering information.

In particular, the baseline depressurization-stopping triggering information is a voice feature, such as “stopping depressurizing” and other voice features, controlled the depressurization device to stop discharging the gas in the confined space of the external auditory meatus. The requirement of the baseline depressurization-stopping triggering information is same as the voice feature corresponding to the baseline depressurization-stopping triggering information.

It is determined whether the operator is necessary to control the depressurization device to stop discharging the gas in the confined space of the external auditory meatus by judging whether the voice feature corresponding to the depressurization-stopping triggering information is same as the voice feature corresponding to the baseline depressurization-stopping triggering information.

Step 6021: continuing to obtain the depressurization-stopping triggering information, if the depressurization-stopping triggering information does not meet the requirement of the preset baseline depressurization-stopping triggering information.

In particular, if the voice feature corresponding to the depressurization-stopping triggering information is different from the voice feature corresponding to the baseline depressurization-stopping triggering information, it means that the operator does not needs to control the depressurization device to stop discharging the gas in the confined space of the external auditory meatus. Therefore, the depressurization-stopping triggering information is continuously to detected, thereby continuing to monitor the changes of the depressurization-stopping triggering information.

Step 6022: controlling the depressurization device to stop depressurization, if the depressurization-stopping triggering information meets the requirement of the preset baseline depressurization-stopping triggering information.

In particular, if the voice feature corresponding to the depressurization-stopping triggering information is same as the voice feature corresponding to the baseline depressurization-stopping triggering information, it means that the operator needs to control the depressurization device to stop discharging the gas in the confined space of the external auditory meatus, thereby controlling the depressurization device to stop depressurization.

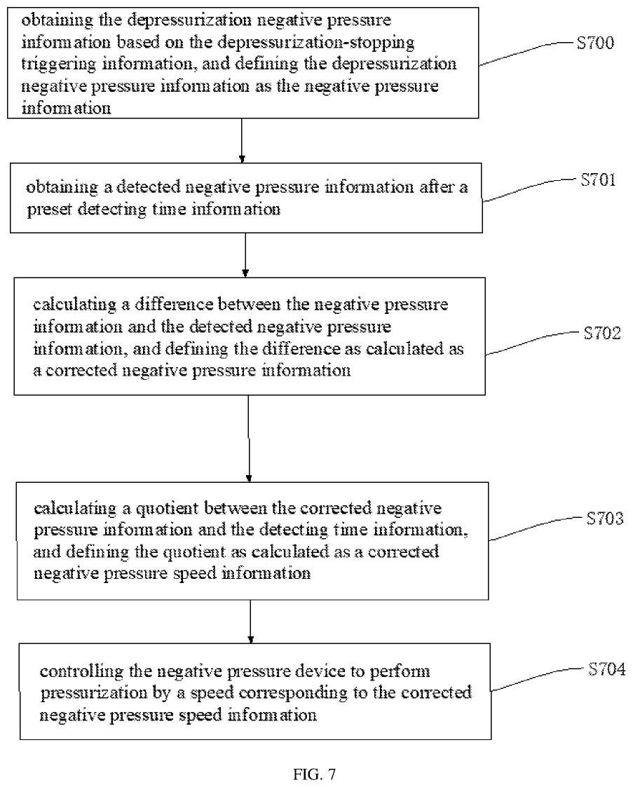

Referring to FIG. 7, when the depressurization device stops depressurization, a control method of the negative pressure information includes following steps:

Step 700: obtaining the depressurization negative pressure information based on the depressurization-stopping triggering information, and defining the depressurization negative pressure information as a negative pressure information.

In particular, the depressurization negative pressure information is a pressure value of the remaining gas in the confined space of the external auditory meatus against the external auditory and the ear drum after the depressurization device discharges the gas in the confined space of the external auditory meatus, which is detected by the force sensitive sensor of the ear plug and sent to the processing device. After detecting the depressurization negative pressure information, the depressurization negative pressure is defined as the negative pressure information.

Step 701: obtaining a detected negative pressure information after a preset detecting time information.

In particular, the detecting time information is a period for detecting the gas pressure value of the confined space of the external auditory meatus, which is set by those skilled in the art according to actual needs. The detected negative pressure information is a pressure value of the gas in the confined space for the ear drum and the external auditory meatus after the period corresponding to the detecting time information, which is detected by the force sensitive sensor of the ear plug and sent to the processing device.

Step 702: calculating a difference between the negative pressure information and the detected negative pressure information, and defining the difference as calculated as a corrected negative pressure information.

In particular, the corrected negative pressure information is a reduced pressure value of the gas in the confined space of the external auditory meatus for the external auditory and the ear drum after the detecting time information, which is obtained by the processing device by calling the detected negative pressure information and the negative pressure information, and calculating a difference between the negative pressure value corresponding to the negative pressure information and the negative pressure value corresponding to the detected negative pressure information.

Step 703: calculating a quotient between a corrected negative pressure information and the detecting time information, and defining the quotient as calculated as a corrected negative pressure speed information.

In particular, the corrective negative spade information is a pressure reduction value of the gas in the confined space of the external auditory meatus for the external auditory meatus and the ear drum in unit time, which is obtained by the processing device calling the corrected negative pressure information and the detecting time information, and calculating the quotient between the negative pressure value corresponding to the corrected negative pressure information and the time value corresponding to the detecting time information.

Step 704: controlling the negative pressure device to perform pressurization by a speed corresponding to the corrected negative pressure speed information.

In particular, the negative pressure device is controlled to supply the negative pressure into the confined space of the external auditory meatus according to the pressure reduction value in unit time corresponding to the corrected negative pressure speed information, thereby maintaining the stability of the negative pressure of the confined space of the external auditory meatus. The specific method for supplying the negative pressure is as follows: when the volume is constant, the negative pressure value is directly proportional to the gas amount, and a gas supplying amount in unit time is calculated according to the reduced negative pressure value between the directly proportional coefficient and the unit, thereby controlling the negative pressure device to deliver the gas corresponding to the gas supplying amount in unit time to the confined space of the external auditory meatus.

Based on the same invention conception, a device according to the present application is disclosed, which is applied any one of the methods for controlling the external ear negative pressure generator in FIGS. 1-7, and includes:

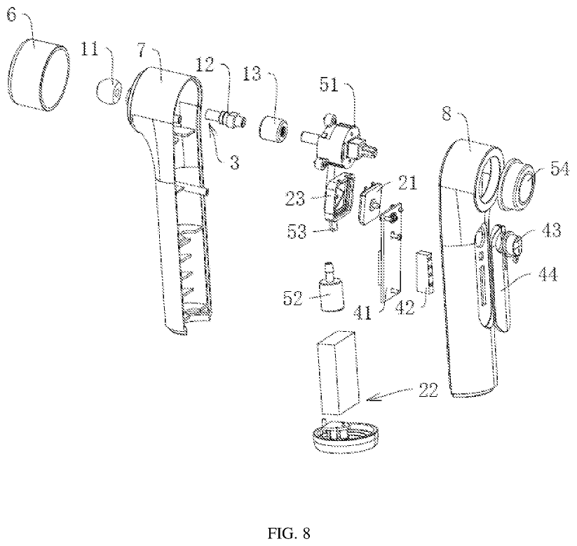

Referring to FIG. 8, an ear plug 1 is inserted in an external auditory meatus to form a confined space where a negative pressure device 2 generates a negative pressure information. The ear plug 1 is installed on a front housing 7, and includes an ear plug 11, an ear plug support 12 and a connection sealing element 13. The ear plug 11 is sleeved on the ear plug support 12, such that the ear plug support 12 supports the ear plug 11, and the ear plug support 12 is in communication with the gas tube 53, such that the gas tube 53 delivers the gas to the ear plug 11 and the external auditory meatus through the ear plug support 12. The connection sealing element 13 is installed between the ear plug support 12 and the gas tube 53. It can be made of rubber, and seals a seam between the ear plug support 12 and the gas tube 53, thereby avoiding the gas leakage.

A negative pressure device 2 is connected to the ear plug 1, and is configured to generate the negative pressure information. The negative pressure device 2 is installed in a mounting cavity defined between the front housing 7 and the back housing 8, the negative pressure device 2 includes a piezoelectric ceramic pump 21, a cell assembly 22 and a negative support pump support 23, and the piezoelectric ceramic pump 21 configured to drive the gas through the gas tube 53 to the ear plug support 12 is connected to the gas tube 53. The negative support pump support 23 configured to fix the piezoelectric ceramic pump 21 is installed on the front housing 7. The cell assembly 22 configured to supply power for the whole device includes a cell and a charging component.

A detecting device 3 configured to detect the negative pressure information is provided on the ear plug 1. The detecting device 3 includes a force sensitive sensor installed on the ear plug support 12 and configured to be detected the pressure of the gas in the confined space of the external auditory meatus.

A processing device 4 is individually electrically connected to the negative pressure device 2 and the detecting device 3, and is configured to compare the negative pressure information with the safety negative pressure, to control the negative pressure device 2 to generate the negative pressure, and to control the detecting device 3 to detect the negative pressure information. The processing device 4 includes a mainboard 41, a shade 42, a button 43 and a translucent panel 44, and the mainboard 41 is configured to control the negative pressure device 2, the detecting device 3 and a depressurization device 5. The shade 42 is installed on the mainboard 41 and is configured to reduce the brightness of the lighting on the mainboard 41, and a shading hole configured to be inserted through by the shade 42 is defined on the back housing 8. The button 43 is installed on the back housing 8, a button hole configured to be inserted by the button 43 is defined on the back housing 8, which enables the button 43 to insert into the button hole, thereby pressing a start switch on the mainboard 41. The translucent panel 44 is installed on a side of the back housing 8 adjacent to the shading hole, and is configured to protect the mainboard 41.

A depressurization device 5 is configured to discharge the gas in the confined space of the external auditory meatus to reduce the negative pressure value. The depressurization device 5 includes a manual valve 51, a safety valve 52, a gas tube 53 and a button cap 54. The manual valve 51 and the safety valve 52 are both installed in the mounting cavity defined between the front housing 7 and the back housing 8, and are both in communication with the gas tube 53. The safety valve 52 can be an electrical valve, which is controlled by the processing device 4, and configured to passively discharge the gas in the confined space of the external auditory meatus to reduce the negative pressure value. The button cap 54 is installed on a side of the back housing 8 adjacent to the manual valve 51, a pressing hole configured to be inserted through by the button cap 54 is defined on the back housing 8. When the operator presses the button cap 54, the button cap 54 abuts against the manual valve 51, thereby opening the manual valve 51 to discharge the gas in the confined space of the external auditory meatus.

The embodiment of the present application provides a computer-readable storage medium, on which a computer program is stored to be loaded and be implemented a method for controlling the external ear negative pressure generator by the processor.

The computer-readable storage medium includes various mediums for storing the program code, such as U disk, mobile hard disk, Read-Only Memory (Read-Only Memory, ROM), Random Access Memory (Random Access Memory, RAM), disk or light disk and so on.

Based on the same invention conception, the embodiment of the present application provides an intelligent terminal including a memory and a processor, and the memory stores a computer program being loaded and being implemented a method for controlling the external ear negative pressure generator by the processor.

The above are the preferred embodiments of the present application, which are not intended to limit the protection scope of the present application. Therefore, all equivalent changes made according to the structure, shape and principle of the present application should be covered within the protection scope of the present application.

### LIST OF REFERENCE SIGNS

1 Ear plug
11 Ear plug
12 Ear plug support
13 Connection sealing element
2 Negative pressure device
21 Piezoelectric ceramic pump
22 Cell assembly
23 Negative support pump support
3 Detecting device
4 Processing device
41 Mainboard
42 Shade
43 Button
44 Translucent panel
5 Depressurization device
51 Manual valve
52 Safety valve
53 Gas tube
54 Button cap
6 Transparent cover
7 Front housing
8 Back housing

What is claimed is:

1. A control method of an external ear negative pressure generator, comprising:
obtaining start information of the external ear negative pressure generator;
obtaining external auditory meatus triggering information based on the start information;
controlling a preset negative pressure device to generate negative pressure information based on the external auditory meatus triggering information, and obtaining pressure detection information of an external auditory meatus;
judging whether a value of the pressure detection information is smaller than or equal to a pressure value corresponding to a preset safety pressure information;
continuing to obtain the pressure detection information when the value of the pressure detection information is smaller than the pressure value corresponding to the preset safety pressure information; and obtaining pressurization triggering information when the value of the pressure detection information is equal to the pressure value corresponding to the preset safety pressure information;
judging whether the pressurization triggering information meets a requirement of preset baseline pressurization triggering information; and
controlling the preset negative pressure device to stop generating the negative pressure information when the pressurization triggering information does not meet the requirement of the preset baseline pressurization triggering information; and controlling the preset negative pressure device to perform pressurization by preset pressurization data information according to the pressurization triggering information when the pressurization triggering information meets the requirement of the preset baseline pressurization triggering information.

2. The control method of the external ear negative pressure generator according to claim 1, wherein a method for controlling the preset negative pressure device to generate the negative pressure information according to the external auditory meatus comprises:
obtaining input negative pressure information based on the external auditory meatus triggering information;
judging whether the input negative pressure information meets a requirement of preset baseline input negative pressure information;
controlling the preset negative pressure device to generate the negative pressure information according to preset input negative pressure step length information when the input negative pressure information does not meet the requirement of the preset baseline input negative pressure information; and judging whether the input negative pressure information is larger than a safety pressure information when the input negative pressure information meets the requirement of the preset baseline input negative pressure information; and
controlling the preset negative pressure device to generate the negative pressure information according to the input negative pressure information when the input negative pressure information is not larger than the safety pressure information; and controlling the preset negative pressure device to generate base negative pressure information according to the safety pressure information, and, based on the base negative pressure information, generating the negative pressure information according to the preset pressurization data information until the negative pressure information is equal to the input negative pressure information when the input negative pressure information is larger than the safety pressure information.

3. The control method of the external ear negative pressure generator according to claim 2, wherein a method for controlling the preset negative pressure device to generate the base negative pressure information according to the safety pressure information comprises:
obtaining normal negative pressure information of the external auditory meatus;
calculating a difference between the base negative pressure information and the normal negative pressure information, and defining the difference as calculated as changed negative pressure information;
obtaining gas capacity information of the external auditory meatus;
analyzing a gas amount corresponding to the gas capacity information and a negative pressure value corresponding to the changed negative pressure information to determine a changed capacity information of the external auditory meatus;
obtaining external auditory meatus diameter information;
analyzing a capacity value corresponding to the changed capacity information and a diameter corresponding to the external auditory meatus diameter information to determine deformation length information of a preset ear plug; and
controlling the preset negative pressure device to drive ear plug to deform by a deformation length corresponding to the deformation length information and compress a gas to generate the base negative pressure information.

4. The control method of the external ear negative pressure generator according to claim 3, wherein a method for controlling the preset negative pressure device to drive the ear plug to deform by the deformation length corresponding to the deformation length information and compress the gas to generate the base negative pressure information comprises:
obtaining external auditory meatus length information;
judging whether the deformation length information is smaller than the external auditory meatus length information;
controlling the preset negative pressure device to deform the ear plug by the deformation length corresponding to the deformation length information and compress the gas to generate the base negative pressure information when the deformation length information is smaller than the external auditory meatus length information; and determining safety deformation length information according to the external auditory meatus length information when the deformation length information is not smaller than the external auditory meatus length information; and
controlling the preset negative pressure device to deform the ear plug and to generate the base negative pressure information according to the safety deformation length information.

5. The control method of the external ear negative pressure generator according to claim 4, wherein a method for controlling the preset negative pressure device to deform the ear plug to generate the base negative pressure information according to the safety deformation length information comprises:
controlling the preset negative pressure device to deform the ear plug by the deformation length corresponding to the safety deformation length information;
obtaining deformation negative pressure information;
calculating a difference between the base negative pressure information and the deformation negative pressure information, and defining the difference as calculated as supplementary negative pressure information;
calculating a difference between the external auditory meatus length information and the safety deformation length information, and defining the difference as calculated as remaining length information;
analyzing the diameter corresponding to the external auditory meatus diameter information and a length corresponding to the remaining length information to determine remaining capacity information;
analyzing a capacity corresponding to the remaining capacity information and a negative pressure corresponding to the supplementary negative pressure information to determine supplementary gas capacity information; and
controlling the preset negative pressure device to deliver the gas corresponding to the supplementary gas capacity information to the external auditory meatus by preset gas input time information to generate the base negative pressure information.

6. The control method of the external ear negative pressure generator according to claim 2, wherein, when the negative pressure information is equal to the input negative pressure information, a depressurizing method of the negative pressure information comprises:
obtaining depressurization triggering information;
judging whether the depressurization triggering information meets a requirement of preset baseline depressurization triggering information;
continuing to obtain the depressurization triggering information when the depressurization triggering information does not meet the requirement of the preset baseline depressurization triggering information; and controlling a depressurization device to depressurize by preset depressurization data information and obtaining depressurization-stopping triggering information when the depressurization triggering information meets the requirement of the preset baseline depressurization triggering information;
judging whether the depressurization-stopping triggering information meets a requirement of preset baseline depressurization-stopping triggering information;
continuing to obtain the depressurization-stopping triggering information when the depressurization-stopping triggering information does not meet the requirement of the preset baseline depressurization-stopping triggering information; and controlling the depressurization device to stop depressurizing when the depressurization-stopping triggering information meets the requirement of the preset baseline depressurization-stopping triggering information.

7. The control method of the external ear negative pressure generator according to claim 6, wherein, when the depressurization device stops depressurization, a control method of the negative pressure information comprises:
obtaining depressurization negative pressure information based on the depressurization-stopping triggering information, and defining the depressurization negative pressure information as the negative pressure information;
obtaining detected negative pressure information after preset detecting time information;
calculating a difference between the negative pressure information and the detected negative pressure information, and defining the difference as calculated as corrected negative pressure information;
calculating a quotient between the corrected negative pressure information and the preset detecting time information, and defining the quotient as calculated as corrected negative pressure speed information; and
controlling the preset negative pressure device to perform pressurization by a speed corresponding to the corrected negative pressure speed information.

8. A device used with the control method of the external ear negative pressure generator in claim 1, comprising:
an ear plug configured to be inserted into the external auditory meatus to form a confined space where the preset negative pressure device generates the negative pressure information;
the preset negative pressure device connected to the ear plug and configured to generate the negative pressure information;
a detecting device provided on the ear plug and configured to detect detected negative pressure information; and
a processing device electrically connected to the preset negative pressure device and the detecting device, respectively, and configured to compare the negative pressure information with safety negative pressure information, and control the preset negative pressure device to generate the negative pressure information and control the detecting device to detect the detected negative pressure information according to a comparison result.

9. An intelligent terminal, comprising a memory and a processor, wherein the memory stores a computer program configured to be loaded with and execute the control method of the external ear negative pressure generator in claim 1 by the processor.
